(19) United States
(12) Patent Application Publication
(10) Pub. No.: US 2025/0120570 A1
(43) Pub. Date: Apr. 17, 2025

(54) BIFLUIDIC VALVES FOR ENDOSCOPES

(71) Applicant: Boston Scientific Medical Device Limited, Galway (IE)

(72) Inventors: Aditya Dhanotiya, Indore (IN); Shrikant Vasant Raut, Mumbai (IN); Pooja Bhuvanesh Kulkarni, Pune (IN); James Weldon, Newton, MA (US)

(73) Assignee: Boston Scientific Medical Device Limited, Galway (IE)

(21) Appl. No.: 18/910,681

(22) Filed: Oct. 9, 2024

Related U.S. Application Data
(60) Provisional application No. 63/589,857, filed on Oct. 12, 2023.

(51) Int. Cl. A61B 1/00; A61B 1/005

(52) U.S. Cl. CPC A61B 1/00068; A61B 1/005

(57) ABSTRACT

An endoscope (e.g., a duodenoscope) includes a bifluidic valve for controlling the flow of a liquid (e.g., water) and a gas (e.g., air) through a flexible tubular probe to respectively irrigate or insufflate an internal cavity of a patient. The bifluidic valve can be a single component, an interconnected assembly of components, or a plurality of separate components. Some examples of the bifluidic valve include a stepped diameter valve spool with radial or circumferential fluid passageways. In some examples, the bifluidic valve includes a tube pincher that can stop the water or other liquid from flowing through a flexible tube by pinching the tube shut. Some example bifluidic valves include a valve housing that is resiliently flexible to urge the valve to a normally closed position. In some examples, selectively covering or uncovering a vent in the valve housing controls the flow of air for insufflation.

### CROSS-REFERENCE TO RELATED APPLICATIONS

This application claims the benefit of U.S. Provisional Patent Application Ser. No. 63/589,857 filed on Oct. 12, 2023, the disclosure of which is incorporated herein by reference.

### TECHNICAL FIELD

Various aspects of this disclosure relate generally to endoscopes (e.g., duodenoscopes, colonoscopes, bronchoscopes, etc.) and more specifically to fluidic valves for endoscopes.

### BACKGROUND

Endoscopes have revolutionized the field of medical diagnostics and interventions by enabling medical practitioners to directly visualize internal cavities of the human body without the need for invasive surgeries. Among the various types of endoscopes, duodenoscopes hold a prominent place due to their capability to explore the upper gastrointestinal tract, particularly the duodenum, pancreas, and bile ducts. Duodenoscopes facilitate not only visual examinations but also a range of therapeutic procedures, making them indispensable tools in modern medicine.

Duodenoscopes typically comprise a flexible tubular probe for inserting into the patient. A light source at a distal end of the probe provides illumination for viewing. Often a high-resolution camera is adjacent to the light source for capturing real-time images or videos of the internal cavities. The camera's capabilities ensure detailed and accurate observations.

A duodenoscope's probe usually includes a working channel, allowing for the insertion of various instruments for procedures like biopsies, tissue removal, or stent placement. To provide maneuverability and access to intricate anatomical structures, many duodenoscope probes have internal wires. The tension in the wires can be adjusted in opposing sets of two by manipulating knobs on a handle body of the duodenoscope. Adjusting the wire tension enables bending and steering of the probe.

Handle bodies typically include valves for controlling fluid flow, such as air for insufflation and water for irrigation. Insufflation is a technique used during endoscopic procedures to improve visualization. It involves the introduction of air or carbon dioxide into the cavity being examined, which helps to expand the space, allowing for better visibility of the targeted area. In duodenoscopy, insufflation aids in the assessment of mucosal surfaces and the identification of abnormalities that might otherwise be obscured. This technique enhances the accuracy of diagnoses and assists in determining appropriate treatment strategies.

Irrigation typically involves the introduction of liquids, such as sterile water or saline. Irrigation serves several purposes during endoscopic procedures. It can help clear blood, debris, or mucus from the visual field, ensuring clear visibility. Irrigation can also aid in therapeutic interventions by flushing out areas of interest, allowing for better access and manipulation of tissues. This feature may enhance the safety and effectiveness of procedures like polyp removal or tissue sampling.

### SUMMARY

The present disclosure generally pertains to endoscopes and methods for using them. In some examples, the endoscope may include a valve housing supported by a handle body, wherein the valve housing includes a liquid inlet, a liquid outlet, a gas inlet and a gas outlet. Some examples, the endoscope may include a valve spool with a first portion and a second portion, wherein the first portion has a gas passageway, and the second portion has a liquid passageway. In some examples, the valve spool is movable between an off position and a irrigate position. In some examples, the valve spool in combination with the valve housing is a bifluidic valve selectively configurable to a vented configuration and an insufflating configuration. In some examples, the liquid passageway connects the liquid inlet in fluid communication with the liquid outlet when the valve spool is in the irrigate position. In some examples, the gas passageway connects the gas inlet in fluid communication with the gas outlet when the bifluidic valve is in the insufflating configuration. Some examples of the endoscope include a flexible tubular probe with a proximal end attached to the handle body and a distal end that can be inserted into the patient. In some examples, the flexible tubular probe connects the liquid outlet and the gas outlet in fluid communication with the distal end. In some examples, the first portion of the valve spool is larger in diameter than the second portion. In some examples, gas passageway and the liquid passageway extend circumferentially around the valve spool. In some examples, gas passageway and the liquid passageway extend radially through the valve spool. In some examples, the valve spool includes a pushbutton head with a vent. In some examples, covering the vent places the valve in an insufflating configuration. In some examples, uncovering the vent places the valve in a vented configuration. In some examples, the valve spool in the irrigate position obstructs fluid communication between the gas inlet and the gas outlet.

In some examples, the endoscope may include a flexible tube connecting the liquid inlet to the liquid outlet. In some examples, the flexible tube is deformable between a deformed shape and a more relaxed shape. In some examples, the flexible tube provides greater flow restriction therethrough when the flexible tube is in the deformed shape than when the flexible tube is in the more relaxed shape. Some examples of the endoscope may include a pincher engaging the flexible tube. In some examples, the pincher is movable between an off position and an irrigate position. In some examples, the pincher in the off position forces the flexible tube to the deformed shape. In some examples, the pincher in the irrigate position restores the flexible tube to the more relaxed shape.

In some examples, using the endoscope involves inserting the flexible tubular probe into the patient, injecting a gas or a liquid through the flexible tubular probe into the patient, pinching the deformable tube to decrease a liquid flow rate of the liquid conveyed to the patient, and manually obstructing a vent to increase a gas flow rate of the gas conveyed to the patient.

The preceding summary is provided to facilitate an understanding of some of the features of the present disclosure and is not intended to be a full description. A full appreciation of the disclosure can be gained by taking the entire specification, claims, drawings and abstract.

In some embodiments, the disclosure can be implemented as an endoscope for use on a patient. The endoscope can comprise a handle body; a valve housing supported by the handle body, the valve housing providing a liquid inlet, a liquid outlet, a gas inlet and a gas outlet; a valve spool having a first portion and a second portion, the first portion at least partially defining a gas passageway, the second portion at least partially defining a liquid passageway, the valve spool being movable relative to the valve housing selectively to an off position and an irrigate position, the valve spool in combination with the valve housing providing a bifluidic valve selectively configurable to a vented configuration and an insufflating configuration; the liquid passageway connecting the liquid inlet in fluid communication with the liquid outlet when the valve spool is in the irrigate position, the gas passageway connecting the gas inlet in fluid communication with the gas outlet when the bifluidic valve is in the insufflating configuration; and a flexible tubular probe with a proximal end attached to the handle body and a distal end being insertable into the patient, the flexible tubular probe connecting the liquid outlet and the gas outlet in fluid communication with the distal end.

In further embodiments of the endoscope, the first portion is larger in diameter than the second portion.

In further embodiments of the endoscope, the gas passageway and the liquid passageway extend circumferentially around the valve spool.

In further embodiments, the endoscope can comprise a pushbutton head on the valve spool, wherein the valve spool and the pushbutton head define a vent, the bifluidic valve being in the insufflating configuration when the vent is covered while the valve spool is in the off position, the bifluidic valve being in the vented configuration when the vent is open, the vent connecting the gas passageway in fluid communication with atmosphere when the bifluidic valve is in the vented configuration, the vent being covered and inhibiting fluid flow through the vent to atmosphere when the bifluidic valve is in the insufflating configuration.

In further embodiments of the endoscope, the valve spool in the irrigate position obstructs fluid communication between the gas inlet and the gas outlet.

In further embodiments of the endoscope, the valve spool in the off position obstructs fluid communication between the liquid inlet and the liquid outlet.

In further embodiments of the endoscope, the gas passageway and the liquid passageway pass through the valve spool.

In further embodiments, the endoscope can comprise a spring urging the valve spool to the off position.

In further embodiments of the endoscope, the valve spool is comprised of a metal and the valve housing is comprised of a polymer.

With some embodiments, the disclosure can be implemented as an endoscope. The endoscope can comprise a handle body; a valve housing supported by the handle body, the valve housing providing a liquid inlet, a liquid outlet, a gas inlet and a gas outlet; a flexible tube connecting the liquid inlet to the liquid outlet, the flexible tube being deformable between a deformed shape and a more relaxed shape, the flexible tube providing greater flow restriction therethrough when the flexible tube is in the deformed shape than when the flexible tube is in the more relaxed shape; a pincher engaging the flexible tube, the pincher being movable between an off position and an irrigate position, the pincher in the off position forces the flexible tube to the deformed shape, the pincher in the irrigate position restores the flexible tube to the more relaxed shape; and a flexible tubular probe with a proximal end attached to the handle body and a distal end being insertable into the patient, the flexible tubular probe connecting the liquid outlet and the gas outlet in fluid communication with the distal end.

In further embodiments, the endoscope can comprise a water inlet conduit; a water outlet conduit extending through the flexible tubular probe; a first joint connecting the water inlet conduit to the flexible tube at the water inlet; and a second joint connecting the water outlet conduit to the flexible tube at the water outlet.

In further embodiments of the endoscope, resilient flexure of the valve housing renders the pincher movable between the off position and the irrigate position.

In further embodiments of the endoscope, resilience of the valve housing urges the pincher to the off position.

In further embodiments of the endoscope, the pincher is seamlessly part of the valve housing, and resilient flexure of the valve housing renders the pincher movable between the off position and the irrigate position.

In further embodiments of the endoscope, the valve housing in combination with the flexible tube and the pincher provides a bifluidic valve, the valve housing provides a gas passageway between the gas inlet and the gas outlet, the valve housing defines a vent connected in fluid communication with the gas passageway, the vent being selectively covered and uncovered by the user, the vent when covered places the bifluidic valve in a insufflating configuration, the valve when uncovered places the bifluidic valve in a vented configuration, the vent connecting the gas passageway in fluid communication with atmosphere when the bifluidic valve is in the vented configuration, the vent being covered and inhibiting fluid flow through the vent to atmosphere when the bifluidic valve is in the insufflating configuration.

In further embodiments of the endoscope, the valve housing is a seamless unitary piece.

In further embodiments, the endoscope can comprise a spring urging the flexible tube to the deformed shape.

With some embodiments, the disclosure can be implemented as a method for using an endoscope on a patient, wherein the endoscope includes a flexible tubular probe, a deformable tube and a valve with a vent. The method can comprise inserting the flexible tubular probe into the patient; injecting selectively a gas and a liquid through the flexible tubular probe into the patient; pinching the deformable tube to decrease a liquid flow rate of the liquid conveyed to the patient; and manually obstructing the vent to increase a gas flow rate of the gas conveyed to the patient.

In further embodiments of the method, the endoscope includes a resiliently flexible valve housing that engages the deformable tube and defines the vent.

In further embodiments, the method can comprise flexing the resiliently flexible valve housing to increase the liquid flow rate.

### BRIEF DESCRIPTION OF THE DRAWINGS

FIG. 1 is a perspective view of an example endoscope incorporating various aspects of the present disclosure.

FIG. 2 is a cross-sectional view of an example valve in an off position while in a vented configuration, wherein the valve includes various aspects of the present disclosure.

FIG. 3 is a cross-sectional view like FIG. 2 but showing the valve in the off position while in an insufflating configuration.

FIG. 4 is a cross-sectional view like FIGS. 2 and 3 but showing the valve in an irrigate position.

FIG. 5 is a cross-sectional view of another example valve in an off position while in a vented configuration, wherein the valve includes various aspects of the present disclosure.

FIG. 6 is a cross-sectional view like FIG. 5 but showing the valve in the off position while in an insufflating configuration.

FIG. 7 is a cross-sectional view like FIGS. 5 and 6 but showing the valve in an irrigate position.

FIG. 8 is a perspective view of another valve in an off position while in a vented configuration, wherein the valve includes various aspects of the present disclosure.

FIG. 9 is a perspective view like FIG. 8 but showing the valve in an irrigate position while in a vented configuration.

FIG. 10 is a perspective view like FIGS. 8 and 9 but showing the valve in the off position while in an insufflating configuration.

FIG. 11 is a cross-sectional view taken along line 11-11 of FIG. 8.

FIG. 12 is a cross-sectional view taken along line 12-12 of FIG. 9.

FIG. 13 is a perspective view of another valve in an off position while in a vented configuration, wherein the valve includes various aspects of the present disclosure.

FIG. 14 is a cross-sectional view taken along line 14-14 of FIG. 13.

FIG. 15 is a cross-sectional view taken along line 15-15 of FIG. 13.

FIG. 16 is a cross-sectional view like FIG. 14 but showing the valve in an off position while in the insufflating configuration.

FIG. 17 is a cross-sectional view like FIG. 15 but showing the valve in the off position while in the insufflating configuration.

FIG. 18 is a cross-sectional view like FIG. 16 but showing the valve in the irrigate position while in the insufflating configuration.

FIG. 19 is a cross-sectional view like FIG. 17 but showing the valve in the irrigate position while in the insufflating configuration.

FIG. 20 is a perspective view of another valve in an off position while in a vented configuration, wherein the valve includes various aspects of the present disclosure.

FIG. 21 is a cross-sectional view taken along line 21-21 of FIG. 20.

FIG. 22 is a cross-sectional view taken along line 22-22 of FIG. 20.

FIG. 23 is a cross-sectional view like FIG. 21 but showing the valve in an off position while in the insufflating configuration.

FIG. 24 is a cross-sectional view like FIG. 22 but showing the valve in the off position while in the insufflating configuration.

FIG. 25 is a cross-sectional view like FIG. 23 but showing the valve in the irrigate position while in the insufflating configuration.

FIG. 26 is a cross-sectional view like FIG. 24 but showing the valve in the irrigate position while in the insufflating configuration.

FIG. 27 is a perspective view of another valve in an off position while in a vented configuration, wherein the valve includes various aspects of the present disclosure.

FIG. 28 is a cross-sectional view taken along line 28-28 of FIG. 27.

FIG. 29 is a front view of FIG. 27

FIG. 30 is a cross-sectional view like FIG. 28 but showing the valve in a closed position while in the insufflating configuration.

FIG. 31 is a front view like FIG. 29 but showing the valve in the closed position while in the insufflating configuration.

FIG. 32 is a cross-sectional view like FIG. 30 but showing the valve in an irrigate position while in the vented configuration.

FIG. 33 is a front view like FIG. 31 but showing the valve in the irrigate position while in the vented configuration.

FIG. 34 is a flow diagram illustrating various method steps for using an endoscope, wherein the method steps involve various aspects of the present disclosure.

### DESCRIPTION

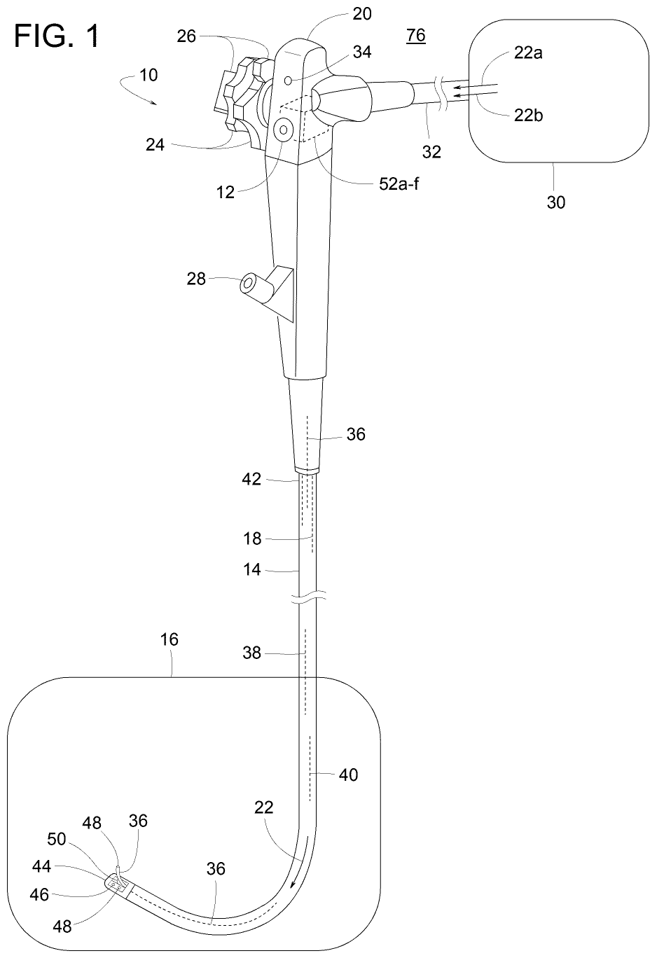

FIGS. 1-34 show various examples of a bifluidic valve 12 for an endoscope 10 and methods for using them. The term, “bifluidic” as it relates to a valve means that the valve can handle at least two streams of fluid, wherein one fluid is a gas, and the other fluid is a liquid. The valve can be a single component, an interconnected assembly of components, or a plurality of separate components. The term, “endoscope” represents any medical apparatus with a flexible tubular probe 14 for inserting into a patient 16 to visually explore the patient's internal tissues and cavities and to introduce water, air, or other fluids when desired. Some example endoscopes 10 have internal wires 18 with adjustable tension for bending and steering the flexible tubular probe 14. Some examples of the endoscope 10, shown in FIG. 1, include duodenoscopes, colonoscopes, ureteroscopes, bronchoscopes, laparoscopes, sheaths, and catheters.

The endoscope 10 is illustrated as an example, so many of the following listed components are optional. Some examples of the endoscope 10 include components such as a handle body 20, the flexible tubular probe 14 extending from the handle body 20, the bifluidic valve 12 (e.g., bifluidic valves 12a-f) for controlling the flow of fluid 22 (e.g., a liquid 22a and a gas 22b), steering knobs 24 to adjust the tension in the internal wires 18, locking levers 26 to lock the position of the steering knobs 24, a biopsy port 28 for sampling withdrawn tissue or fluid, a control unit 30, an umbilicus 32 connecting the control unit 30 to the handle body 20, and an image capture button 34.

The control unit 30 provides various functions. Some examples of such functions include supplying liquid 22a (e.g., water), supplying gas 22b (e.g., air), sending and receiving electrical signals, processing electrical signals, providing a source of vacuum, etc. Some of the control unit's functions listed here are optional. The umbilicus 32 connects the control unit 30 in signal communication or fluid communication with the bifluidic valve 12, the flexible tubular probe 14, or other endoscope-related components.

In some examples, the flexible tubular probe 14 contains various components such as the internal wires 18 for steering, tubing 36 (one or more tubes) for conveying fluids 22, a fiber optic cable 38 for conveying images or light, and electrical wires 40 for conveying electrical power or signals. Some of these probe components are optional.

The flexible tubular probe 14 has a proximal end 42 and a distal end 44. The proximal end 42 connects to the handle body 20, and the distal end 44 extends away from the handle body 20. At the distal end 44, some examples of the flexible tubular probe 14 include a light 46 (or fiber optic cable leading thereto) for illuminating a patient's internal cavities, a camera 48 (or fiber optic cable leading thereto), a tip 48 of the tubing 36, and an elevator 50 for tilting the tip 48 of the tubing 36. The elevator 50 is also known as a swing stand, a pivot stand, and a raising bed. The tip 48 of the tubing 36 is open to pass fluid 22 for insufflating, irrigating, or biopsy sampling.

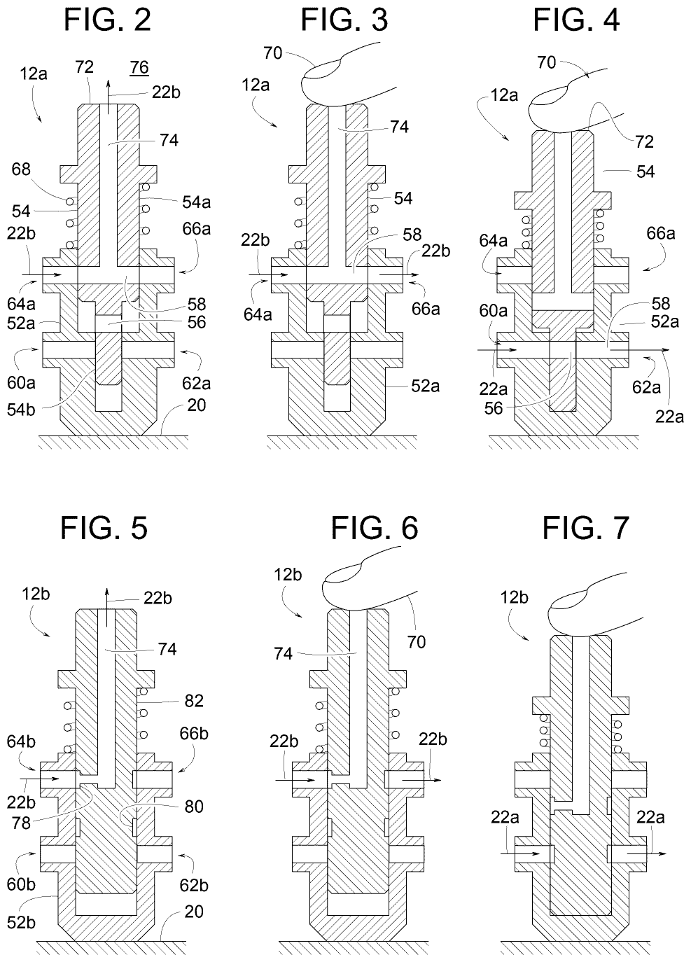

Insufflating and irrigating can be controlled by various examples of the bifluidic valve 12 (e.g., bifluidic valves 12a-f). In the example shown in FIGS. 2-4, the bifluidic valve 12a comprises a valve housing 52a and a valve spool 54. The valve housing 52a is supported by the handle body 20, as shown in FIGS. 2-4. The means for support is shown schematically, as the valve housing 52a can be connected in any way and at any location and orientation relative to the handle body 20. In some examples, the valve housing 52a is a seamless integral extension of the handle body 20.

The valve housing 52a is shown as a seamless unitary piece (i.e., a single part rather than an assembly of parts); however, other examples of valve housing 52a comprise a plurality of parts. In some examples, the plurality parts are interconnected as an assembly. In some examples, the plurality of parts are separated and spaced apart from each other. In some examples, the valve spool 54 is made of metal (e.g., stainless steel, brass, aluminum, etc.) while the valve housing 52a is made of a polymer such as, for example, nylon, PEEK (polyetheretherketone), POM (polyoxymethylene, polyacetal, Delrin, Celcon, etc.), POM-C (polyoxymethylene copolymer), and POM-H (polyoxymethylene homopolymer). Such a combination of metal and polymeric materials can provide an accurate fit and low friction between the valve spool 54 and the valve housing 52a.

In the example shown in FIGS. 2-4, the valve spool 54 includes a first portion 54a and a second portion 54b. The second portion 54b at least partially defines a gas passageway 56. The first portion 54b at least partially defines a liquid passageway 58. The valve housing 52a defines or otherwise provides a liquid inlet 60a, a liquid outlet 62a, a gas inlet 64a, and a gas outlet 66a.

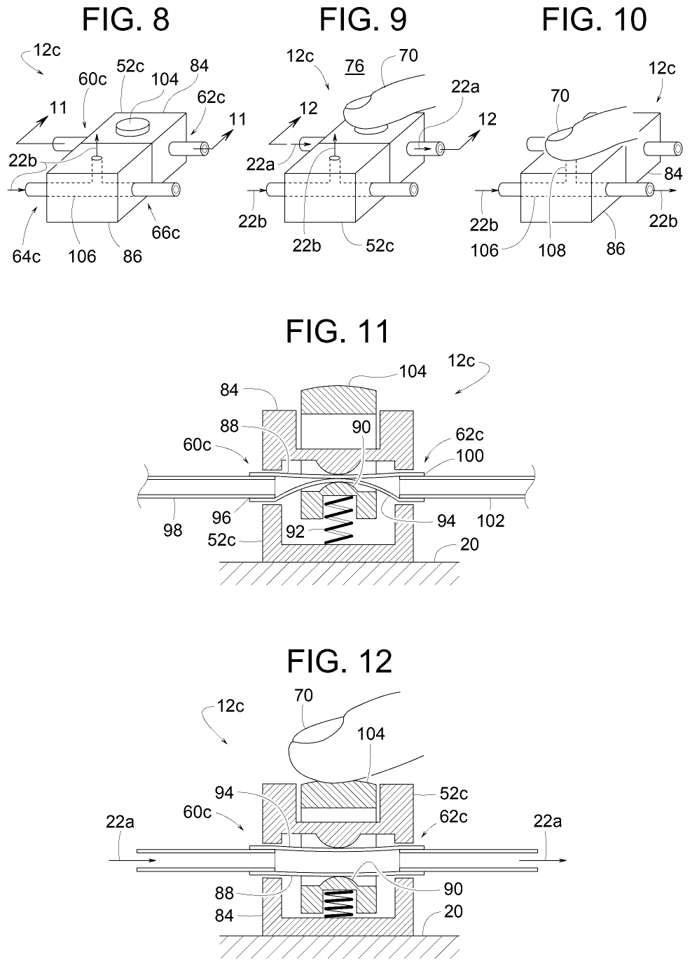

The terms inlet and outlet refer to openings through which a fluid can pass. A valve housing providing an inlet, or an outlet means that the valve housing has an inlet or outlet opening through which a fluid can pass; however, the fluid does not necessarily have to contact the valve housing or contact the inner wall of the opening. For instance, in some examples, as shown in FIGS. 8-33, a tube conveying a fluid might extend through an inlet opening in the valve housing, so the fluid flows through the tube and touches the inner wall of the tube. Naturally, the fluid flowing through the tube also passes through the inlet opening in the valve housing but does not actually touch the inner wall of the valve housing.

The liquid inlet 60a of valve housing 52a receives liquid 22a (e.g., water) from the control unit 30 via umbilicus 32. The tubing 36 within the flexible tubular probe 14 connects the liquid outlet 62a to the probe's distal end 44.

The gas inlet 64a of the valve housing 52a receives gas 22b (e.g., air) from the control unit 30 via umbilicus 32. The gas outlet 66a is connected to discharge the gas 22b through the flexible tubular probe 14 to the probe's distal end 44.

To control the flow of liquid 22a through the flexible tubular probe 14, the valve spool 54 is movable within the valve housing 52a between an off position (FIGS. 2 and 3) and an irrigate position (FIG. 4). In some examples, a spring 68 urges the valve spool 54 to the off position. In the off position, the liquid passageway 56 is misaligned with the liquid inlet 60a and liquid outlet 62b, such that the valve spool 54 blocks liquid flow through the bifluidic valve 12a.

To initiate liquid flow, a user 70 (e.g., a medical practitioner) depresses the valve spool 54 to align the liquid passageway 56 with the liquid inlet 60a and liquid outlet 60b, thereby placing the valve spool 54 in the irrigate position (FIG. 4). In the irrigate position, the liquid 22a flows in series from the liquid inlet 60a, through the liquid passageway 56, through the liquid outlet 62a, through the tubing 36 in the flexible tubular probe 14, and into the patient 16. In some examples, as shown in FIG. 4, moving the valve spool 54 to the irrigate position is done by the user 70 pressing their finger against a pushbutton head 72 of the valve spool 54. The term, “pushbutton head” simply refers to a part that is accessible to the user 70 for affecting the operation of the valve. In FIGS. 2-4, the pushbutton head 72 is the exposed end of the valve spool 54.

In some examples, the bifluidic valve 12a is further configurable selectively to a vented configuration (FIG. 2) and an insufflating configuration (FIG. 3). In the vented configuration, a vent 74 passing through the pushbutton head 72 of the valve spool 54 is left uncovered and open to connect the gas passageway 58 in fluid communication with atmosphere 76. When the gas passageway 58 is vented to atmosphere 76, the gas 22b from the gas inlet 64a has a much greater tendency to escape through the vent 74 than to flow through the gas outlet 66a and the tubing 36 in the flexible tubular probe 14.

To insufflate or promote the flow of gas 22b to the patient 16, the user 70 can cover the vent 74, as shown in FIG. 3. This places the bifluidic valve 12a in its insufflating configuration. When the vent 74 is covered while the valve spool 54 is in the off position, as shown in FIG. 3, the gas 22b from gas inlet 64a cannot escape through the vent 74, so the gas 22b from the gas inlet 64a flows in series through the gas passageway 58, through the gas outlet 66a, through the tubing 36 in the flexible tubular probe 14, and into the patient 16.

Although FIG. 4 shows the vent 74 covered, the bifluidic valve 12a is not in the insufflating configuration because the valve spool 54 is depressed, which misaligns the gas passageway 58 with the valve inlet 64a and valve outlet 66a. Thus, the valve spool 54, in the position shown in FIG. 4, blocks the flow of gas 22b through the bifluidic valve 12a.

In some examples, as shown in FIGS. 2-4, the first portion 54a of the valve spool 54 is larger in outer diameter than the second portion 54b. During manufacturing of the valve spool 54, a turning operation on a lathe can produce undesirable run-out (out of roundness) of a relatively long workpiece cantilevered from the jaws of the lathe. This problem can be minimized by turning the second portion 54b of the valve spool 54 at a smaller diameter.

Like the bifluidic valve 12a, the bifluidic valve 12b, shown in FIGS. 5-7, includes a valve housing 52b supported by the handle body 20, wherein the valve housing 52b defines or otherwise provides a liquid inlet 60b, a liquid outlet 62b, a gas inlet 64b, and a gas outlet 66b. While the gas and liquid passageways 58 and 56 of the bifluidic valve 12a, shown in FIGS. 2-4, pass in a radial direction through the valve spool 54, FIGS. 5-7 show the bifluidic valve 12b with a gas passageway 78 and a liquid passageway 80 that extend circumferentially around a valve spool 82. The vent 74 is in fluid communication with the gas passageway 78. Having the gas and liquid passageways 78 and 80 run circumferentially rather than radially might reduce localized areas of relatively high fluid pressure and thus reduce leakage. The function of bifluidic valves 12a and 12b are otherwise the same with FIGS. 5, 6 and 7 corresponding to FIGS. 2, 3 and 4, respectively.

Although the valve spool 82, shown in FIGS. 5-7, does not have a stepped diameter for minimizing run-out, this problem can be minimized by machining the valve spool 82 on a Swiss-style lathe. Instead of supporting the workpiece in a cantilevered manner from the chuck of a more conventional lathe, a Swiss lathe has a bushing for supporting the workpiece in a radial direction, right near where metal removal occurs. During the cut, the workpiece can be slid axially along the bushing, so cutting tool forces occur adjacent to the bushing, rather than farther down the length of a cantilevered workpiece.

FIGS. 8-12 show the example bifluidic valve 12c, which comprises a valve housing 52c supported by the handle body 20 and providing a liquid inlet 60c, a liquid outlet 62c, a gas inlet 64c, and a gas outlet 66c. In some examples, the liquid inlet 60c of the valve housing 52c receives liquid 22a from the control unit 30 via umbilicus 32. In some examples, the tubing 36 within the flexible tubular probe 14 connects the liquid outlet 62c to the probe's distal end 44. In some examples, the tubing 36 that runs through the flexible tubular probe 14 also extends through the liquid inlet 60c and the liquid outlet 62c of the valve housing 52c.

In some examples, the valve housing 52c includes a first portion 84 for handling the liquid 22a and a second portion 86 for handling the gas 22b. In some examples, the two portions 84 and 86 are combined as a seamless unitary piece. In some examples, the two portions 84 and 86 are discrete interconnected parts. In some examples, the two portions 84 and 86 are separated and spaced apart.

In some examples of endoscope 10, the first portion 84 of the valve housing 52c is supported by the handle body 20, as schematically illustrated in FIGS. 11 and 12. In some examples, the valve housing 52c holds a flexible tube 88, a pincher 90, and a compression spring 92.

In some examples, the flexible tube 88 extends between the liquid inlet 60c and the liquid outlet 62c. In some examples, the flexible tube 88 is a seamless integral part of the tubing 36 that runs through the flexible tubular probe 14 and/or through the umbilicus 32. This eliminates joints and possible points of leakage.

In other examples, the flexible tube 88 is a relatively short segment 94 with a first joint 96 connecting the flexible tube 88 to a water inlet conduit 98 and with a second joint 100 connecting the flexible tube 88 to a water outlet conduit 102. The short segment 94 can be designed with physical and material attributes that make the flexible tube 88 more durable and tolerant of repeated deformation, while water inlet and outlet conduits 98 and 102 can be made more suitable for use in a relatively long flexible tubing probe 14.

In some examples, the water outlet conduit 102 is the tubing 36 that runs through the flexible tubular probe 14. In some examples, the water inlet conduit 98 runs through the umbilicus 32. Regardless of whether the flexible tube 88 is a short segment connected to the water inlet and outlet conduits 98 and 102 or is a seamless integral part of the tubing 36 that runs through the flexible tubular probe 14, the flexible tube 88 is deformable between a deformed shape (FIG. 11) and a more relaxed shape (FIG. 12). The flexible tube 88 provides greater flow restriction therethrough when the flexible tube 88 is in the deformed shape (FIG. 11) than when the flexible tube 88 is in the more relaxed shape (FIG. 12).

To move the flexible tube 88 from its deformed shape to its more relaxed shape, and thereby open the valve, the user 70 can press on a pushbutton head 104 of the pincher 90. This moves the pincher 90 from an off position (FIG. 11) to an irrigate position (FIG. 12).

In some examples when the user 70 releases the pushbutton head 104, the compression spring 92 pushes the pincher 90 back to its off position, as shown in FIG. 11. In other examples, without the compression spring 92, resilience of the valve housing 52c itself is what urges the pincher 90 to its off position. In some examples, the pincher 90 is seamlessly part of the valve housing 52c, and the resilient flexure of the valve housing 52c renders the pincher 90 movable between the off position (FIG. 11) and the irrigate position (FIG. 12).

As for the second portion 86 of the valve housing 52c, the gas inlet 64c receives gas 22b from the control unit 30 via umbilicus 32. A gas passageway 106 connects the gas inlet 64c in fluid communication with the gas outlet 66c. The gas outlet 66c is connected to discharge the gas 22b through the flexible tubular probe 14 to the probe's distal end 44.

In some examples, a vent 108 connects the gas passageway 106 to atmosphere 76. The user 70 can cover or uncover the vent 108, which makes the bifluidic valve 12c configurable selectively to a vented configuration (FIGS. 8 and 9) and an insufflating configuration (FIG. 10). In the vented configuration, the gas 22b from the gas inlet 64c has a much greater tendency to escape through the vent 108 than to flow through the gas outlet 66c and the tubing 36 in the flexible tubular probe 14.

To insufflate or promote the flow of gas 22b to the patient 16, the user 70 can cover the vent 108, as shown in FIG. 10. This places the bifluidic valve 12c in its insufflating configuration. When the vent 108 is covered, the gas 22b has a greater tendency to flow in series from the gas inlet 64c, through the gas passageway 106, through the gas outlet 66c, through the tubing 36 in the flexible tubular probe 14, and into the patient 16.

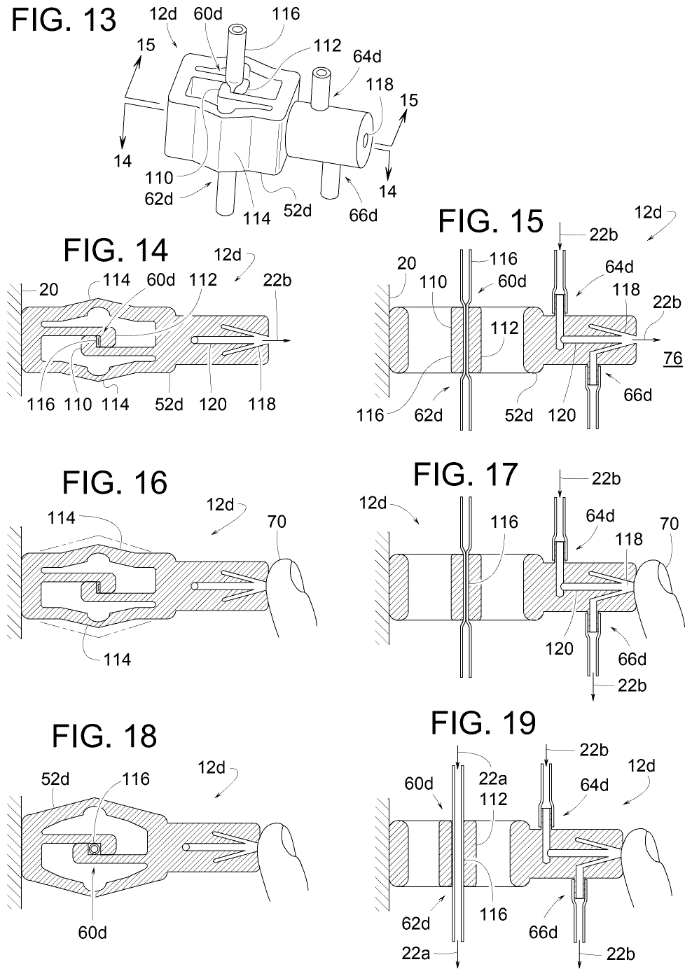

FIGS. 13-19 show the example bifluidic valve 12d, which comprises a valve housing 52d supported by the handle body 20. The means for support is shown schematically, as the valve housing 52d can be connected in any way and at any location and orientation relative to the handle body 20. In some examples, the valve housing 52d is a seamless integral extension of the handle body 20.

In some examples, the valve housing 52d comprises a pincher 110, a platen 112, at least one spring member 114, and a flexible tube 116 lying between the pincher 110 and the platen 112. In some examples, the valve housing 52d defines or otherwise provides a liquid inlet 60d and a liquid outlet 62d, both of which are between the pincher 110 and the platen 112. The liquid inlet 60d connects to the umbilicus 32 to receive liquid 22a from the control unit 30, and the liquid outlet 62d is connected in fluid communication with the flexible tubular probe 14 to deliver liquid 22a to the probe's distal end 44.

In some examples, the flexible tube 116 lying between the pincher 110 and the platen 112 is an integral extension of the tubing 36 running through the flexible tubular probe 14 and/or is an integral extension of tubing running through umbilicus 32, thereby minimizing the number of tubing joints and possible points of leakage. In some examples, the flexible tube 116 lying between the pincher 110 and the platen 112 is a relatively short, more durable segment like the short segment 94 shown in FIGS. 11 and 12.

The flexibility of the spring member 114 allows the pincher 110 to be moved resiliently between an off position (FIGS. 13-17) and an irrigate position (FIGS. 18 and 19). In response to the pincher 110 moving between the off position and the irrigate position, the flexible tube 116 deforms between a deformed shape (FIGS. 13-17) and a more relaxed shape (FIGS. 18 and 19), respectively. The liquid 22a can flow through the bifluidic valve 12d when the flexible tube 116 is in the more relaxed shape in the irrigate position. The bifluidic valve 12d blocks the flow of liquid 22a when the flexible tube 116 is in the deformed shape while in the off position.

To open the liquid portion of the bifluidic valve 12d to the irrigate position, the user 70 compresses the valve housing 52d from the position shown in FIGS. 16 and 17 to that of FIGS. 18 and 19. Flexure of spring member 114 allows the pincher 110 to move away from the platen 112, thereby creating a space therebetween in which the flexible tube 116 can relax and thus open by expanding radially.

To close the liquid portion of the bifluidic valve 12d to the off position, the user 70 simply releases the manual pressure against the valve housing 52d. With the pressure from the user 70 removed, the resilience of the spring member 114 urges the valve housing 52d back to its normally closed off position, as shown in FIGS. 14 and 15.

In some examples, the valve housing 52d also defines or otherwise provides a gas inlet 64d, a gas outlet 66d, and a vent 118. To supply the valve housing 52d with gas 22b, the umbilicus 32 connects the gas inlet 64d to the control unit 30. A gas passageway 120 connects the gas inlet 64d in fluid communication with the gas outlet 66d. The gas outlet 66d is connected to discharge the gas 22b through the flexible tubular probe 14 to the probe's distal end 44.

In some examples, the vent 118 connects the gas passageway 120 to atmosphere 76. The user 70 can cover or uncover the vent 118, which makes the bifluidic valve 12d configurable selectively to a vented configuration (FIGS. 13-15) and an insufflating configuration (FIGS. 16-19). In the vented configuration, the gas 22b from the gas inlet 64d has a much greater tendency to escape through the vent 118 than to flow through the gas outlet 66d and the tubing 36 in the flexible tubular probe 14. The tendency for the gas 22b to escape through the open vent 118 is even greater when vent 118 and gas passageway 120 are laid out in a more tortuous pattern, as shown in FIGS. 14-19.

To insufflate or promote the flow of gas 22b to the patient 16, the user 70 can cover the vent 118, as shown in FIGS. 16-19. This places the bifluidic valve 12d in its insufflating configuration. When the vent 118 is covered, the gas 22b has a greater tendency to flow in series from the gas inlet 64d, through the gas passageway 120, through the gas outlet 66d, through the tubing 36 in the flexible tubular probe 14, and into the patient 16.

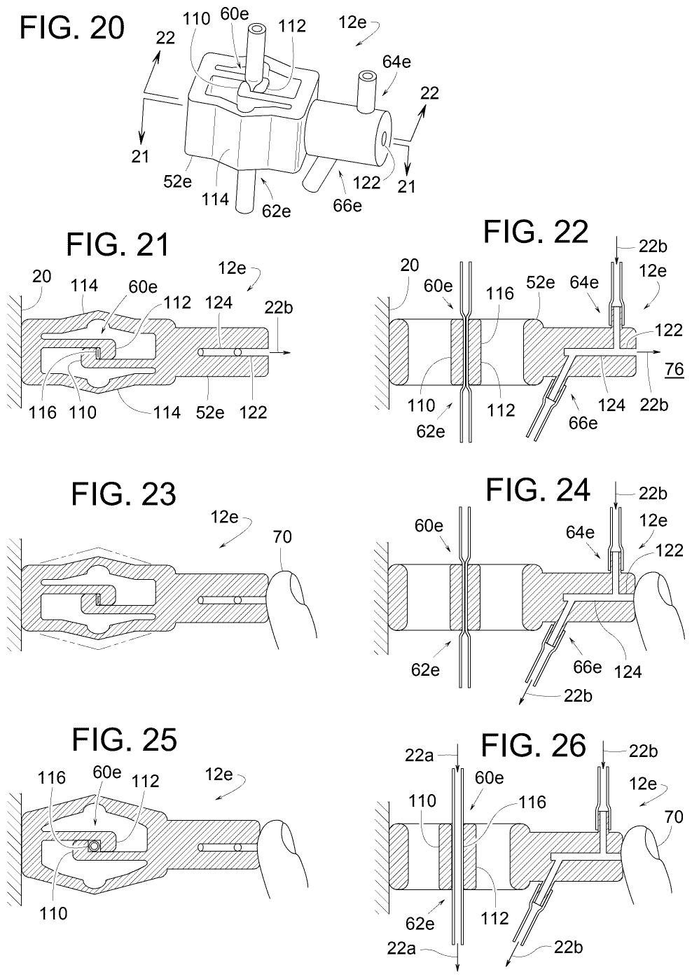

FIGS. 20-26 show the example bifluidic valve 12e, which comprises a valve housing 52e supported by the handle body 20. The means for support is shown schematically, as the valve housing 52e can be connected in any way and at any location and orientation relative to the handle body 20. In some examples, the valve housing 52e is a seamless integral extension of the handle body 20.

In some examples, the valve housing 52e comprises the pincher 110, the platen 112, at least one spring member 114, and the flexible tube 116 lying between the pincher 110 and the platen 112. In some examples, the valve housing 52e defines or otherwise provides a liquid inlet 60e and a liquid outlet 62e, both of which are between the pincher 110 and the platen 112. The liquid inlet 60e connects to the umbilicus 32 to receive liquid 22a from the control unit 30, and the liquid outlet 62e is connected in fluid communication with the flexible tubular probe 14 to deliver liquid 22a to the probe's distal end 44.

In some examples, the flexible tube 116 lying between the pincher 110 and the platen 112 is an integral extension of the tubing 36 running through the flexible tubular probe 14 and/or is an integral extension of tubing running through umbilicus 32, thereby minimizing the number of tubing joints and possible points of leakage. In some examples, the flexible tube 116 lying between the pincher 110 and the platen 112 is a relatively short, more durable segment like the short segment 94 shown in FIGS. 11 and 12.

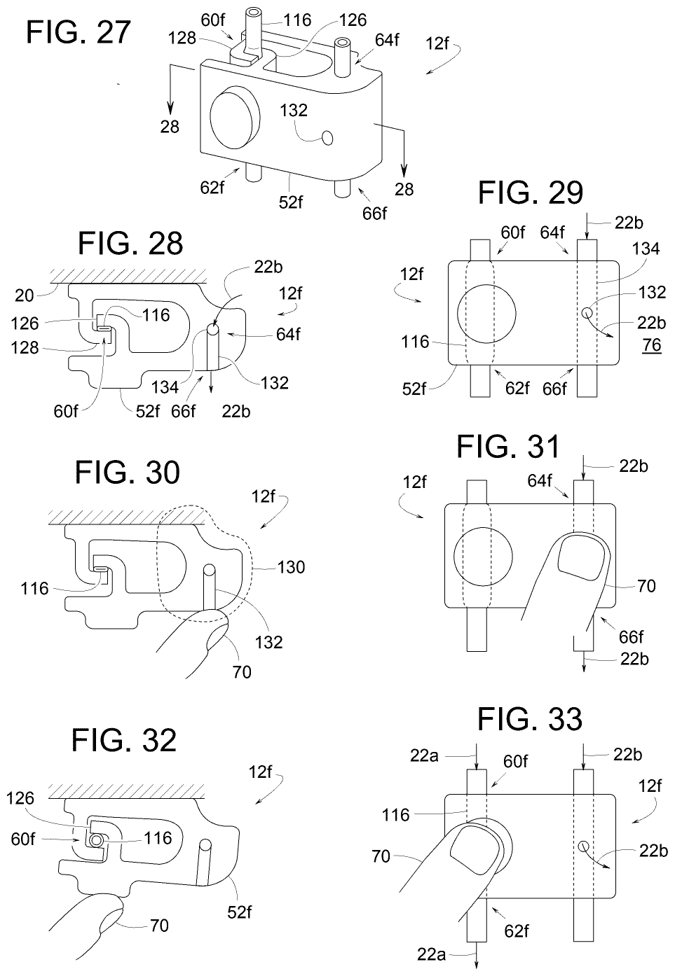

The flexibility of the spring member 114 allows the pincher 110 to be moved resiliently between an off position (FIGS. 20-24) and an irrigate position (FIGS. 25 and 26). In response to the pincher 110 moving between the off position and the irrigate position, the flexible tube 116 deforms between a deformed shape (FIGS. 20-24) and a more relaxed shape (FIGS. 25 and 26), respectively. The liquid 22a can flow through the bifluidic valve 12e when the flexible tube 116 is in the more relaxed shape while in the irrigate position. The bifluidic valve 12e blocks the flow of liquid 22a when the flexible tube 116 is in the deformed shape while in the off position.

To open the liquid portion of the bifluidic valve 12e to the irrigate position, the user 70 compresses the valve housing 52e from the position shown in FIGS. 23 and 24 to that of FIGS. 25 and 26. Flexure of spring member 114 allows the pincher 110 to move away from the platen 112, thereby creating a space therebetween which the flexible tube 116 can relax and thus open by expanding radially.

To close the liquid portion of the bifluidic valve 12e to the off position, the user 70 simply releases the manual pressure against the valve housing 52e. With the pressure from the user 70 removed, the resilience of the spring member 114 urges the valve housing 52e back to its normally closed off position, as shown in FIGS. 21 and 22.

In some examples, the valve housing 52e also defines or otherwise provides a gas inlet 64e, a gas outlet 66e, and a vent 122. To supply the valve housing 52e with gas 22b, the umbilicus 32 connects the gas inlet 64e to the control unit 30. A gas passageway 124 connects the gas inlet 64e in fluid communication with the gas outlet 66e. The gas outlet 66e is connected to discharge the gas 22b through the flexible tubular probe 14 to the probe's distal end 44.

In some examples, the vent 122 connects the gas passageway 124 to atmosphere 76. The user 70 can cover or uncover the vent 122, which makes the bifluidic valve 12e configurable selectively to a vented configuration (FIGS. 20-22) and an insufflating configuration (FIGS. 23-26). In the vented configuration, the gas 22b from the gas inlet 64e has a much greater tendency to escape through the vent 122 than to flow through the gas outlet 66e and the tubing 36 in the flexible tubular probe 14. The tendency for the gas 22b to escape through the open vent 122 is even greater when the relative orientation of the gas inlet 64e and the gas outlet 66e, with respect to the direction of gas flow therethrough, are both angularly offset (not parallel) and laterally offset (not collinear), as shown in FIGS. 21-26.

To insufflate or promote the flow of gas 22b to the patient 16, the user 70 can cover the vent 122, as shown in FIGS. 23-26. This places the bifluidic valve 12e in its insufflating configuration. When the vent 122 is covered, the gas 22b has a greater tendency to flow in series from the gas inlet 64e, through the gas passageway 124, through the gas outlet 66e, through the tubing 36 in the flexible tubular probe 14, and into the patient 16.

FIGS. 27-33 show the example bifluidic valve 12f, which comprises a valve housing 52f supported by the handle body 20. The means for support is shown schematically, as the valve housing 52f can be connected in any way and at any location and orientation relative to the handle body 20. In some examples, the valve housing 52f is a seamless integral extension of the handle body 20.

In some examples, the valve housing 52f comprises a pincher 126, a platen 128, at least one spring member 130, and the flexible tube 116 lying between the pincher 126 and the platen 128. In some examples, the valve housing 52f defines or otherwise provides a liquid inlet 60f and a liquid outlet 62f, both of which are between the pincher 126 and the platen 128. The liquid inlet 60f connects to the umbilicus 32 to receive liquid 22a from the control unit 30, and the liquid outlet 62f is connected in fluid communication with the flexible tubular probe 14 to deliver liquid 22a to the probe's distal end 44.

In some examples, the flexible tube 116 lying between the pincher 126 and the platen 128 is an integral extension of the tubing 36 running through the flexible tubular probe 14 and/or is an integral extension of tubing running through umbilicus 32, thereby minimizing the number of tubing joints and possible points of leakage. In some examples, the flexible tube 116 lying between the pincher 126 and the platen 128 is a relatively short, more durable segment like the short segment 94 shown in FIGS. 11 and 12.

In some examples, the valve housing 52f is resiliently flexible, and the spring member 130 is a seamless integral part of the valve housing 52f itself. The flexibility of the spring member 130 allows the pincher 126 to be moved resiliently between an off position (FIGS. 27-31) and an irrigate position (FIGS. 32 and 33). In response to the pincher 126 moving between the off position and the irrigate position, the flexible tube 116 deforms between a deformed shape (FIGS. 27-31) and a more relaxed shape (FIGS. 32 and 33), respectively. The liquid 22a can flow through the bifluidic valve 12f when the flexible tube 116 is in the more relaxed shape while in the irrigate position. The bifluidic valve 12f blocks the flow of liquid 22a when the flexible tube 116 is in the deformed shape while in the off position.

To open the liquid portion of the bifluidic valve 12f to the irrigate position, the user 70 compresses the valve housing 52f from the position shown in FIGS. 28 and 29 to that of FIGS. 32 and 33. Flexure of spring member 130 allows the pincher 126 to move away from the platen 128, thereby creating a space therebetween which the flexible tube 116 can relax and thus open by expanding radially.

To close the liquid portion of the bifluidic valve 12f to the off position, the user 70 simply releases the manual pressure against the valve housing 52f. With the pressure from the user 70 removed, the resilience of the spring member 130 urges the valve housing 52f back to its normally closed off position, as shown in FIGS. 28 and 29.

In some examples, the valve housing 52f also defines or otherwise provides a gas inlet 64f, a gas outlet 66f, and a vent 132. To supply the valve housing 52f with gas 22b, the umbilicus 32 connects the gas inlet 64f to the control unit 30. A gas passageway 134 connects the gas inlet 64f in fluid communication with the gas outlet 66f. The gas outlet 66f is connected to discharge the gas 22b through the flexible tubular probe 14 to the probe's distal end 44.

In some examples, the vent 132 connects the gas passageway 134 to atmosphere 76. The user 70 can cover or uncover the vent 132, which makes the bifluidic valve 12f configurable selectively to a vented configuration (FIGS. 27-29, 32 and 33) and an insufflating configuration (FIGS. 30-31). In the vented configuration, the gas 22b from the gas inlet 64f has a much greater tendency to escape through the vent 132 than to flow through the gas outlet 66f and the tubing 36 in the flexible tubular probe 14.

To insufflate or promote the flow of gas 22b to the patient 16, the user 70 can cover the vent 132, as shown in FIGS. 30 and 31. This places the bifluidic valve 12f in its insufflating configuration. When the vent 132 is covered, the gas 22b has a greater tendency to flow in series from the gas inlet 64f, through the gas passageway 134, through the gas outlet 66f, through the tubing 36 in the flexible tubular probe 14, and into the patient 16.

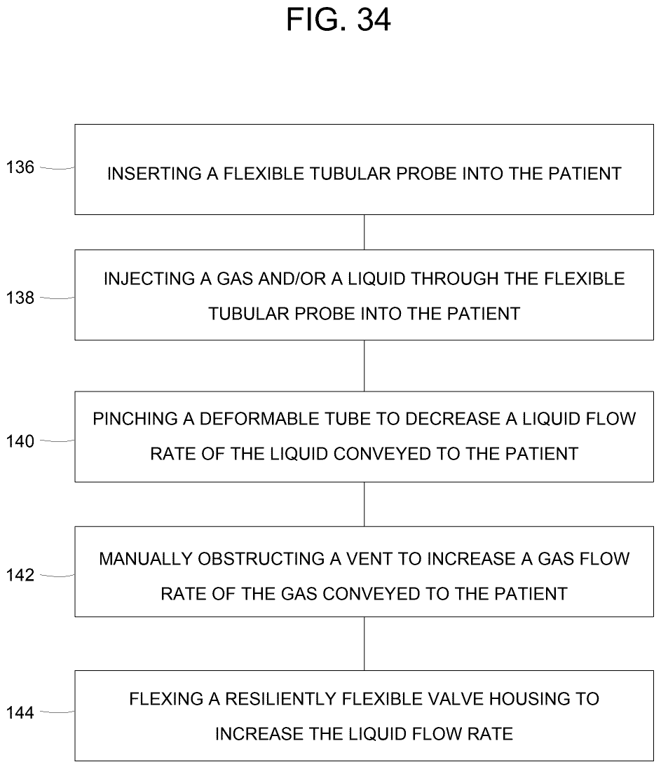

FIG. 34 shows some example method steps for using various examples of the endoscope 10. The method steps shown in FIG. 34 do necessarily have to be performed in any order, and not all the method steps necessarily have to be performed. A block 136 represents inserting the flexible tubular probe 14 into the patient 16. A block 138 represents injecting selectively a gas 22b and a liquid 22a through the flexible tubular probe 14 into the patient 116. A block 140 represents pinching the deformable tube (e.g., deformable tubes 88 or 116) to decrease a liquid flow rate of the liquid 22a conveyed to the patient 16. A block 142 represents manually obstructing the vent (e.g., vents 74, 108, 118, 122 or 132) to increase a gas flow rate of the gas 22b conveyed to the patient 16. A block 144 represents flexing the resiliently flexible valve housing (e.g., valve housings 52c-f) to increase the liquid flow rate.

The disclosure should not be considered limited to the examples described above. Various modifications, equivalent processes, as well as numerous structures to which the disclosure can be applicable will be readily apparent to those of ordinary skill in the art upon review of the instant specification.

1. An endoscope for use on a patient comprising:
a handle body;
a valve housing supported by the handle body, the valve housing providing a liquid inlet, a liquid outlet, a gas inlet and a gas outlet;
a valve spool having a first portion and a second portion, the first portion at least partially defining a gas passageway, the second portion at least partially defining a liquid passageway, the valve spool being movable relative to the valve housing selectively to an off position and an irrigate position, the valve spool in combination with the valve housing providing a bifluidic valve selectively configurable to a vented configuration and an insufflating configuration;
the liquid passageway connecting the liquid inlet in fluid communication with the liquid outlet when the valve spool is in the irrigate position, the gas passageway connecting the gas inlet in fluid communication with the gas outlet when the bifluidic valve is in the insufflating configuration; and
a flexible tubular probe with a proximal end attached to the handle body and a distal end being insertable into the patient, the flexible tubular probe connecting the liquid outlet and the gas outlet in fluid communication with the distal end.

2. The endoscope of claim 1, wherein the first portion is larger in diameter than the second portion.

3. The endoscope of claim 1, wherein the gas passageway and the liquid passageway extend circumferentially around the valve spool.

4. The endoscope of claim 1, further comprising a pushbutton head on the valve spool, wherein the valve spool and the pushbutton head define a vent, the bifluidic valve being in the insufflating configuration when the vent is covered while the valve spool is in the off position, the bifluidic valve being in the vented configuration when the vent is open, the vent connecting the gas passageway in fluid communication with atmosphere when the bifluidic valve is in the vented configuration, the vent being covered and inhibiting fluid flow through the vent to atmosphere when the bifluidic valve is in the insufflating configuration.

5. The endoscope of claim 1, wherein the valve spool in the irrigate position obstructs fluid communication between the gas inlet and the gas outlet.

6. The endoscope of claim 1, wherein the valve spool in the off position obstructs fluid communication between the liquid inlet and the liquid outlet.

7. The endoscope of claim 1, wherein the gas passageway and the liquid passageway pass through the valve spool.

8. The endoscope of claim 1, further comprising a spring urging the valve spool to the off position.

9. The endoscope of claim 1, wherein the valve spool is comprised of a metal and the valve housing is comprised of a polymer.

10. An endoscope for a user to use on a patient comprising:
a handle body;
a valve housing supported by the handle body, the valve housing providing a liquid inlet, a liquid outlet, a gas inlet and a gas outlet;
a flexible tube connecting the liquid inlet to the liquid outlet, the flexible tube being deformable between a deformed shape and a more relaxed shape, the flexible tube providing greater flow restriction therethrough when the flexible tube is in the deformed shape than when the flexible tube is in the more relaxed shape;
a pincher engaging the flexible tube, the pincher being movable between an off position and an irrigate position, the pincher in the off position forces the flexible tube to the deformed shape, the pincher in the irrigate position restores the flexible tube to the more relaxed shape; and
a flexible tubular probe with a proximal end attached to the handle body and a distal end being insertable into the patient, the flexible tubular probe connecting the liquid outlet and the gas outlet in fluid communication with the distal end.

11. The endoscope of claim 10, further comprising:
a water inlet conduit;
a water outlet conduit extending through the flexible tubular probe;
a first joint connecting the water inlet conduit to the flexible tube at the water inlet; and
a second joint connecting the water outlet conduit to the flexible tube at the water outlet.

12. The endoscope of claim 10, wherein resilient flexure of the valve housing renders the pincher movable between the off position and the irrigate position.

13. The endoscope of claim 10, wherein resilience of the valve housing urges the pincher to the off position.

14. The endoscope of claim 10, wherein the pincher is seamlessly part of the valve housing, and resilient flexure of the valve housing renders the pincher movable between the off position and the irrigate position.

15. The endoscope of claim 10, wherein the valve housing in combination with the flexible tube and the pincher provides a bifluidic valve, the valve housing provides a gas passageway between the gas inlet and the gas outlet, the valve housing defines a vent connected in fluid communication with the gas passageway, the vent being selectively covered and uncovered by the user, the vent when covered places the bifluidic valve in a insufflating configuration, the valve when uncovered places the bifluidic valve in a vented configuration, the vent connecting the gas passageway in fluid communication with atmosphere when the bifluidic valve is in the vented configuration, the vent being covered and inhibiting fluid flow through the vent to atmosphere when the bifluidic valve is in the insufflating configuration.

16. The endoscope of claim 15, wherein the valve housing is a seamless unitary piece.

17. The endoscope of claim 16, further comprising a spring urging the flexible tube to the deformed shape.

18. A method for using an endoscope on a patient, wherein the endoscope includes a flexible tubular probe, a deformable tube and a valve with a vent, the method comprising:
inserting the flexible tubular probe into the patient;
injecting selectively a gas and a liquid through the flexible tubular probe into the patient;
pinching the deformable tube to decrease a liquid flow rate of the liquid conveyed to the patient; and
manually obstructing the vent to increase a gas flow rate of the gas conveyed to the patient.

19. The method of claim 18, wherein the endoscope includes a resiliently flexible valve housing that engages the deformable tube and defines the vent.

20. The method of claim 19, further comprising flexing the resiliently flexible valve housing to increase the liquid flow rate.
